(19) United States
(12) Patent Application Publication
(10) Pub. No.: US 2003/0170775 A1
(43) Pub. Date: Sep. 11, 2003

(54) Method for modifying the genome of corynebacteria

(76) Inventors: Markus Pompejus, Freinsheim (DE); Hartwig Schroder, Nussloch (DE); Burkhard Kroger, Limburgerhof (DE); Oskar Zelder, Speyer (DE)

Correspondence Address: NIXON & VANDERHYE, PC, 1100 N GLEBE ROAD, 8TH FLOOR, ARLINGTON, VA, 22201-4714, US

(21) Appl. No.: 10/380,179

(22) PCT Filed: Sep. 19, 2001
(86) PCT No.: PCT/EP01/10805

(30) Foreign Application Priority Data
Sep. 20, 2000 (DE) 100 46 870.5

(51) Int. Cl. C12P 25/00; C12P 13/04; C12P 13/08; C12N 1/21; C12N 15/74

(52) U.S. Cl. 435/66; 435/69.1; 435/106; 435/115; 435/320.1; 435/471; 435/252.3

(57) ABSTRACT

The invention relates to a process for producing corynebacteria comprising one or more modified genomic sequences, where a vector is used which does not replicate in corynebacteria and whose nucleic acid is not recognized by corynebacteria as foreign.

The invention relates to a novel process for modifying the genome of corynebacteria, to the use of these bacteria and to novel vectors. In particular, the invention relates to a process for modifying corynebacteria with the aid of vectors which cannot replicate in corynebacteria.

Corynebacterium glutamicum is a Gram-positive aerobic bacterium which (like other corynebacteria, i.e. Corynebacterium and Brevibacterium species) is used in industry for producing a series of fine chemicals, and also for breaking down hydrocarbons and for oxidizing terpenoids (for a review, see, for example, Liebl (1992) “The Genus Corynebacterium”, in: The Procaryotes, Volume II, Balows, A. et al., eds. Springer).

Owing to the availability of cloning vectors for use in corynebacteria and techniques for the genetic manipulation of C. glutamicum and related Corynebacterium and Brevibacterium species (see, for example, Yoshihama et al., J. Bacteriol. 162 (1985) 591-597; Katsumata et al., J. Bacteriol. 159 (1984) 306-311; and Santamaria et al. J. Gen. Microbiol. 130 (1984) 2237-2246) it is possible to genetically modify these organisms (for example by overexpressing genes) in order to make them better and more efficient as producers of one or more fine chemicals.

The use of plasmids which can replicate in corynebacteria is a well-established technique with which the skilled worker is familiar and which is used widely and documented repeatedly in the literature (see, for example, Deb, J. K et al. (1999) FEMS Microbiol. Lett. 175, 11-20).

It is also possible to genetically modify corynebacteria by modifying the DNA sequence of the genome. DNA sequences may be introduced into the genome (they can be newly introduced and/or further copies of existing sequences can be introduced), or else DNA sequence segments can be removed from the genome (for example genes or portions of genes), or else sequence substitutions (for example base substitutions) can be carried out in the genome.

The genome can be modified by introducing, into the cell, DNA which preferably does not replicate in the cell, and by recombination of this DNA which has been introduced with genomic host DNA, thus modifying the genomic DNA. However, the methods known for this purpose are complicated, and all entail specific problems (see, for example, van der Rest, M. E. et al. (1999) Appl. Microbiol. Biotechnol. 52, 541-545).

A known method is based on conjugation (Schwarzer & Pühler (1991) Biotechnology 9, 84-87). The disadvantage is that specific mobilisable plasmids must be used, and these plasmids must be transferred from a donor strain (as a rule E. coli) to the recipient (for example, Corynebacterium species) by conjugation. Moreover, this method is very laborious.

The disadvantages of conjugation are the reason why it is advantageous to carry out, instead of conjugation, the established simple electroporation method (Liebl et al. (1989) FEMS Microbiol Lett. 65, 299-304) in order to modify genomic sequences (and not only in order to introduce freely replicating plasmids). A novel method allowing this has been described (van der Rest, M. E. et al. (1999) Appl. Microbiol. Biotechnol. 52, 541-545); however, this method has other problems. The cells to be transformed are grown at suboptimal low temperatures, specific media additives which adversely affect growth are added to the growth medium, and the cells are treated with a heat shock.

All methods of transferring DNA into corynebacteria share the problem of the restriction system of the corynebacterial host, which digests DNA which it recognizes as foreign. A large number of approaches exists in the literature to avoid this restriction system, but all of these approaches have specific problems.

There are attempts to employ DNA from E. coli strains which carry mutations in the dam and dcm genes (Ankri et al. (1996) Plasmid 35, 62-66). This leads to DNA which no longer carries Dam and Dcm methylation, but continues to possess the E. coli-specific hsd methylation. Corynebacterium continues to recognize this DNA as foreign DNA.

One possibility of circumventing problems with the restriction system is to isolate restriction-deficient mutants (Liebl et al. (1989) FEMS Microbiol Lett. 65, 299-304). However, the disadvantage is that one is restricted to such specific mutant strains.

Another possibility is temporarily to switch off the restriction system, for example by heat shock. This allows a desired effect to be achieved in conjugation (Schwarzer & Pühler (1991) Biotechnology 9, 84-87) and also in electroporation (van der Rest, M. E. et al. (1999) Appl. Microbiol. Biotechnol. 52, 541-545). Disadvantages are the complicated procedure and the effect that the heat shock affects not only the restriction system, but also a large number of other cellular processes. In general, the heat shock response in bacteria, as a reaction to the heat shock, has a multiplicity of consequences for the metabolism of the cells (see, for example, Gross, C. A. (1996), pp. 1382-1399 in Escherichia coli and Salmonella (Neidhart et al., eds.) ASM press, Washington).

For the purposes of the invention, corynebacteria are to be 10 understood as meaning Corynebacterium species, Brevibacterium species and Mycobacterium species. Preferred are Corynebacterium species and Brevibacterium species. Examples of Corynebacterium species and Brevibacterium species are: Brevibacterium brevis, Brevibacterium lactofermentum, Corynebacterium ammoniagenes, Corynebacterium glutamicum, Corynebacterium diphtheriae and Corynebacterium lactofermentum. Examples of Mycobacterium species are: Mycobacterium tuberculosis, Mycobacterium leprae and Mycobacterium bovis.

The following strains stated in the table are particularly preferred:

TABLE
Corynebacterium and Brevibacterium strains:

| Genus | Species | ATCC | FERM | NRRL | CECT | NCIMB | |
|---|---|---|---|---|---|---|---|
| Brevibacterium | ammoniagenes | 21054 | | | | | |
| Brevibacterium | ammoniagenes | 19350 | | | | | |
| Brevibacterium | ammoniagenes | 19351 | | | | | |
| Brevibacterium | ammoniagenes | 19352 | | | | | |
| Brevibacterium | ammoniagenes | 19353 | | | | | |
| Brevibacterium | ammoniagenes | 19354 | | | | | |
| Brevibacterium | ammoniagenes | 19355 | | | | | |
| Brevibacterium | ammoniagenes | 19356 | | | | | |
| Brevibacterium | ammoniagenes | 21055 | | | | | |
| Brevibacterium | ammoniagenes | 21077 | | | | | |
| Brevibacterium | ammoniagenes | 21553 | | | | | |
| Brevibacterium | ammoniagenes | 21580 | | | | | |
| Brevibacterium | ammoniagenes | 39101 | | | | | |
| Brevibacterium | butanicum | 21196 | | | | | |
| Brevibacterium | divaricatum | 21792 | P928 | | | | |
| Brevibacterium | flavum | 21474 | | | | | |
| Brevibacterium | flavum | 21129 | | | | | |
| Brevibacterium | flavum | 21518 | | | | | |
| Brevibacterium | flavum | | | B11474 | | | |
| Brevibacterium | flavum | | | B11472 | | | |
| Brevibacterium | flavum | 21127 | | | | | |
| Brevibacterium | flavum | 21128 | | | | | |
| Brevibacterium | flavum | 21427 | | | | | |
| Brevibacterium | flavum | 21475 | | | | | |
| Brevibacterium | flavum | 21517 | | | | | |
| Brevibacterium | flavum | 21528 | | | | | |
| Brevibacterium | flavum | 21529 | | | | | |
| Brevibacterium | flavum | | | B 11477 | | | |
| Brevibacterium | flavum | | | B 11478 | | | |
| Brevibacterium | flavum | 21127 | | | | | |
| Brevibacterium | flavum | | | B 11474 | | | |
| Brevibacterium | healii | 15527 | | | | | |
| Brevibacterium | ketoglutamicum | 21004 | | | | | |
| Brevibacterium | ketoglutamicum | 21089 | | | | | |
| Brevibacterium | ketosoreductum | 21914 | | | | | |
| Brevibacterium | lactofermentum | | | | 70 | | |
| Brevibacterium | lactofermentum | | | | 74 | | |
| Brevibacterium | lactofermentum | | | | 77 | | |
| Brevibacterium | lactofermentum | 21798 | | | | | |
| Brevibacterium | lactofermentum | 21799 | | | | | |
| Brevibacterium | lactofermentum | 21800 | | | | | |
| Brevibacterium | lactofermentum | 21801 | | | | | |
| Brevibacterium | lactofermentum | | | B11470 | | | |
| Brevibacterium | lactofermentum | | | B11471 | | | |
| Brevibacterium | lactofermentum | 21086 | | | | | |
| Brevibacterium | lactofermentum | 21420 | | | | | |
| Brevibacterium | lactofermentum | 21086 | | | | | |
| Brevibacterium | lactofermentum | 31269 | | | | | |
| Brevibacterium | linens | 9174 | | | | | |
| Brevibacterium | linens | 19391 | | | | | |
| Brevibacterium | linens | 8377 | | | | | |
| Brevibacterium | paraffinolyticum | | | | | 11160 | |
| Brevibacterium | spec. | | | | | | CBS 717.73 |
| Brevibacterium | spec. | | | | | | CBS 717.73 |
| Brevibacterium | spec. | 14604 | | | | | |
| Brevibacterium | spec. | 21860 | | | | | |
| Brevibacterium | spec. | 21864 | | | | | |
| Brevibacterium | spec. | 21865 | | | | | |
| Brevibacterium | spec. | 21866 | | | | | |
| Brevibacterium | spec. | 19240 | | | | | |
| Corynebacterium | acetoacidophilum | 21476 | | | | | |
| Corynebacterium | acetoacidophilum | 13870 | | | | | |
| Corynebacterium | acetoglutamicum | | | B 11473 | | | |
| Corynebacterium | acetoglutamicum | | | B 11475 | | | |
| Corynebacterium | acetoglutamicum | 15806 | | | | | |
| Corynebacterium | acetoglutamicum | 21491 | | | | | |
| Corynebacterium | acetoglutamicum | 31270 | | | | | |
| Corynebacterium | acetophilum | | | B3671 | | | |
| Corynebacterium | ammoniagenes | 6872 | | | | | NCTC 2399 |
| Corynebacterium | ammoniagenes | 15511 | | | | | |
| Corynebacterium | fujiokense | 21496 | | | | | |
| Corynebacterium | glutamicum | 14067 | | | | | |
| Corynebacterium | glutamicum | 39137 | | | | | |
| Corynebacterium | glutamicum | 21254 | | | | | |
| Corynebacterium | glutamicum | 21255 | | | | | |
| Corynebacterium | glutamicum | 31830 | | | | | |
| Corynebacterium | glutamicum | 13032 | | | | | |
| Corynebacterium | glutamicum | 14305 | | | | | |
| Corynebacterium | glutamicum | 15455 | | | | | |
| Corynebacterium | glutamicum | 13058 | | | | | |
| Corynebacterium | glutamicum | 13059 | | | | | |
| Corynebacterium | glutamicum | 13060 | | | | | |
| Corynebacterium | glutamicum | 21492 | | | | | |
| Corynebacterium | glutamicum | 21513 | | | | | |
| Corynebacterium | glutamicum | 21526 | | | | | |
| Corynebacterium | glutamicum | 21543 | | | | | |
| Corynebacterium | glutamicum | 13287 | | | | | |
| Corynebacterium | glutamicum | 21851 | | | | | |
| Corynebacterium | glutamicum | 21253 | | | | | |
| Corynebacterium | glutamicum | 21514 | | | | | |
| Corynebacterium | glutamicum | 21516 | | | | | |
| Corynebacterium | glutamicum | 21299 | | | | | |
| Corynebacterium | glutamicum | 21300 | | | | | |
| Corynebacterium | glutamicum | 39684 | | | | | |
| Corynebacterium | glutamicum | 21488 | | | | | |
| Corynebacterium | glutamicum | 21649 | | | | | |
| Corynebacterium | glutamicum | 21650 | | | | | |
| Corynebacterium | glutamicum | 19223 | | | | | |
| Corynebacterium | glutamicum | 13869 | | | | | |
| Corynebacterium | glutamicum | 21157 | | | | | |
| Corynebacterium | glutamicum | 21158 | | | | | |
| Corynebacterium | glutamicum | 21159 | | | | | |
| Corynebacterium | glutamicum | 21355 | | | | | |
| Corynebacterium | glutamicum | 31808 | | | | | |
| Corynebacterium | glutamicum | 21674 | | | | | |
| Corynebacterium | glutamicum | 21562 | | | | | |
| Corynebacterium | glutamicum | 21563 | | | | | |
| Corynebacterium | glutamicum | 21564 | | | | | |
| Corynebacterium | glutamicum | 21565 | | | | | |
| Corynebacterium | glutamicum | 21566 | | | | | |
| Corynebacterium | glutamicum | 21567 | | | | | |
| Corynebacterium | glutamicum | 21568 | | | | | |
| Corynebacterium | glutamicum | 21569 | | | | | |
| Corynebacterium | glutamicum | 21570 | | | | | |
| Corynebacterium | glutamicum | 21571 | | | | | |
| Corynebacterium | glutamicum | 21572 | | | | | |
| Corynebacterium | glutamicum | 21573 | | | | | |
| Corynebacterium | glutamicum | 21579 | | | | | |
| Corynebacterium | glutamicum | 19049 | | | | | |
| Corynebacterium | glutamicum | 19050 | | | | | |
| Corynebacterium | glutamicum | 19051 | | | | | |
| Corynebacterium | glutamicum | 19052 | | | | | |
| Corynebacterium | glutamicum | 19053 | | | | | |
| Corynebacterium | glutamicum | 19054 | | | | | |
| Corynebacterium | glutamicum | 19055 | | | | | |
| Corynebacterium | glutamicum | 19056 | | | | | |
| Corynebacterium | glutamicum | 19057 | | | | | |
| Corynebacterium | glutamicum | 19058 | | | | | |
| Corynebacterium | glutamicum | 19059 | | | | | |
| Corynebacterium | glutamicum | 19060 | | | | | |
| Corynebacterium | glutamicum | 19185 | | | | | |
| Corynebacterium | glutamicum | 13286 | | | | | |
| Corynebacterium | glutamicum | 21515 | | | | | |
| Corynebacterium | glutamicum | 21527 | | | | | |
| Corynebacterium | glutamicum | 21544 | | | | | |
| Corynebacterium | glutamicum | 21492 | | | | | |
| Corynebacterium | glutamicum | | | B8183 | | | |
| Corynebacterium | glutamicum | | | B8182 | | | |
| Corynebacterium | glutamicum | | | B12416 | | | |
| Corynebacterium | glutamicum | | | B12417 | | | |
| Corynebacterium | glutamicum | | | B12418 | | | |
| Corynebacterium | glutamicum | | | B11476 | | | |
| Corynebacterium | glutamicum | 21608 | | | | | |
| Corynebacterium | lilium | | P973 | | | | |
| Corynebacterium | nitrilophilus | 21419 | | | | 11594 | |
| Corynebacterium | spec. | | P4445 | | | | |
| Corynebacterium | spec. | | P4446 | | | | |
| Corynebacterium | spec. | 31088 | | | | | |
| Corynebacterium | spec. | 31089 | | | | | |
| Corynebacterium | spec. | 31090 | | | | | |
| Corynebacterium | spec. | 31090 | | | | | |
| Corynebacterium | spec. | 31090 | | | | | |
| Corynebacterium | spec. | 15954 | | | | | DSMZ 20145 |
| Corynebacterium | spec. | 21857 | | | | | |
| Corynebacterium | spec. | 21862 | | | | | |
| Corynebacterium | spec. | 21863 | | | | | |

ATCC: American Type Culture Collection, Rockville, MD, USA
FERM: Fermentation Research Institute, Chiba, Japan
NRRL: ARS Culture Collection, Northern Regional Research Laboratory, Peoria, IL, USA
CECT: Coleccion Espanola de Cultivos Tipo, Valencia, Spain
NCIMB: National Collection of Industrial and Marine Bacteria Ltd., Aberdeen, UK
CBS: Centraalbureau voor Schimmelcultures, Baam, NL
NCTC: National Collection of Type Cultures, London, UK
DSMZ: Deutsche Sammiung von Mikroorganismen and Zellkulturen, Braunschweig, Germany

The invention discloses a novel and simple method of modifying genomic sequences in corynebacteria. This may take the form of genomic integrations of nucleic acid molecules (for example complete genes), disruptions (for example deletions or integrative disruptions) and sequence modifications (for example simple or multiple point mutations, complete gene substitutions). The above-described problems do not exist here. The method according to the invention does not depend on the use of specific recipient strains and only requires the normally used cell cultivation and transformation methods.

The Corynebacterium glutamicum cglIM gene was described Schafer et al. (Gene 203, 1997, 93-101). This gene encodes a DNA methyl transferase. In addition, a method is described for increasing the yield of C. glutamicum transformants with the aid of the cglIM gene when using replicative plasmids.

It has been found that methyl transferases, in particular the cglIM gene, can also be used for integrating DNA into the genome of Corynebacterium glutamicum, for example to disrupt or overexpress genes in the genome. This is also possible with other methyl transferases which introduce the corynebacteria-specific methylation pattern. A vector which is not capable of replication in the corynebacterium to be transformed is used for this purpose. A vector which is not capable of replication is to be understood as meaning a DNA which cannot replicate freely in corynebacteria. It is possible that this DNA can replicate freely in other bacteria if it carries, for example, a suitable origin of replication. However, it is also possible that this DNA cannot replicate even in other bacteria, for example when a linear DNA is inserted.

The process according to the invention is based on a direct transformation of C. glutamicum (for example by electroporation) without it being necessary to use specific methods of growing the cells to be transformed or particular transformation methods (such as heat shock and the like).

The transformation can also be carried out with the addition of restriction endonucleases (as described in DE19823834).

The advantage of the process according to the invention is that the DNA which is introduced is not recognized as foreign DNA and is therefore not digested by the restriction system.

A further advantage of the process according to the invention is that no conjugation has to be carried out; this considerably reduces the labor involved and makes possible an improved flexibility when choosing the plasmids employed.

A further advantage is that no specific corynebacterial strains have to be employed and that no specific treatment of the strains to be transformed is necessary; in particular, no heat shock is necessary. For experimental details, see the example.

The mutants generated thus can then be used for producing fine chemicals or, in the case of C. diphtheriae, for the production of, for example, vaccines comprising attenuated or nonpathogenic pathogens. Fine chemicals are to be understood as meaning: organic acids, proteinogenic and nonproteinogenic amino acids, nucleotides and nucleosides, lipids and fatty acids, diols, carbohydrates, aromatic compounds, vitamins, cofactors and enzymes.

The term “fine chemical” is known in the art and comprises molecules which are produced by an organism and used in various fields of industry, such as, for example, the pharmaceuticals industry, the agricultural industry and the cosmetics industry, but is not limited thereto. These compounds comprise organic acids such as tartaric acid, itaconic acid and diaminopimelic acid, proteinogenic and non-proteinogenic amino acids, purine and pyrimidine bases, nucleosides and nucleotides (for example as described in Kuninaka, A. (1996) Nucleotides and related compounds, pp. 561-612, in Biotechnology Vol. 6, Rehm et al., Ed. VCH Weinheim and the references contained therein), lipids, saturated and unsaturated fatty acids (for example arachidonic acid), diols, for example propanediol and butanediol), carbohydrates (for example hyaluronic acid and trehalose), aromatic compounds (for example aromatic amines, vanillin and indigo), vitamins and cofactors (as described in Ullmann's Encyclopedia of Industrial Chemistry, Vol. A27, “Vitamins”, pp. 15 443-613 (1996) VCH Weinheim and the references cited therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held on Sep. 1 to 3, 1994, in Penang, Malaysia, AOCS Press (1995)), enzymes, polyketides (Cane et al. (1998) Science 282: 63-68), and all other chemicals described by Gutcho (1983) in Chemicals by Fermentation, Noyes Data Corporation, ISBN: 0818805086 and the references cited therein. The metabolism and the uses of certain fine chemicals are illustrated in greater detail hereinbelow.

A. Amino Acid Metabolism and Uses

The amino acids comprise the basic structural units of all proteins and are thus essential for the normal cell functions. The term “amino acid” is known in the art. The proteinogenic amino acids, of which 20 kinds exist, act as structural units for proteins in which they are linked to each other via peptide bonds, whereas the nonproteinogenic amino acids (of which hundreds are known) do not usually occur in proteins (see Ullmann's Encyclopedia of Industrial Chemistry, Vol. A2, pp. 57-97 VCH Weinheim (1985)). The amino acids can exist in the D or L configuration, even though L-amino acids are usually the only type which is found in naturally occurring proteins. Biosynthetic pathways and catabolic pathways of each of the 20 proteinogenic amino acids are well characterized in both prokaryotic and eukaryotic cells (see, for example, Stryer, L. Biochemistry, 3rd Edition (1988), p. 578-590). The “essential” amino acids (histidine, isoleucine, leucine, lysine, methionine, phenylalanine, threonine, tryptophan and valine), termed thus since, owing to the complexity of their biosynthyeses, they must be taken up with the food, are converted by simple biosynthetic pathways into the remaining 11 “nonessential” amino acids (alanine, arginine, asparagine, aspartate, cysteine, glutamate, glutamine, glycine, proline, serine and tyrosine). Higher animals are capable of synthesizing some of these amino acids, but the essential amino acids must be taken up with the food for normal protein synthesis to take place.

Apart from their function in protein biosynthesis, these amino acids are interesting chemicals per se, and it has been found that they are used in many different applications in the food, feed, chemical, cosmetics, agricultural and pharmaceuticals industries. Lysine is an important amino acid not only for human nutrition, but also for monograstic animals such as poultry and pigs. Glutamate is used most frequently as a flavor additive (monosodium glutamate, MSG) and widely in the food industry, as are aspartate, phenylalanine, glycine and cysteine. Glycine, L-methionine and tryptophan are all used in the pharmaceuticals industry. Glutamine, valine, leucine, isoleucine, histidine, arginine, proline, serine and alanine are used in the pharmaceuticals and cosmetics industries. Threonine, tryptophan and D/L methionine are widely used feed additives (Leuchtenberger, W. (1996) Amino acids—technical production and use, pp. 466-502 in Rehm et al., (Ed.) Biotechnology Vol. 6, Chapter 14a, VCH Weinheim). It has been found that these amino acids are furthermore suitable as precursors for the synthesis of synthetic amino acids and proteins, such as N-acetylcysteine, S-carboxymethyl-L-cysteine, (S)-5-hydroxytryptophan and other substances described in Ullmann's Encyclopedia of Industrial Chemistry, Vol. A2, pp. 57-97, VCH Weinheim, 1985.

The biosynthesis of these natural amino acids in organisms capable of producing them, for example bacteria, has been characterized thoroughly (for a review of bacterial amino acid biosynthesis and its regulation, see Umbarger, H. E. (1978) Ann. Rev. Biochem. 47: 533-606). Glutamate is synthesized by reductively aminating α-ketoglutarate, an intermediate in the citric acid cycle. Glutamine, proline and arginine are produced in each case in succession starting from glutamate. Serine is biosynthesized in a 3-step process and starts with 3-phosphoglycerate (an intermediate in glycolysis) and results in this amino acid after oxidation, transamination and hydrolysis steps. Cysteine and glycine are produced in each case starting from serine, the former by condensing homocysteine with serine and the latter by transferring the side-chain β-carbon atom to tetrahydrofolate, in a reaction which is catalyzed by serine transhydroxymethylase. Phenylalanine and tyrosine are synthesized from the precursors of the glycolysis and pentose phosphate pathways, erythrose-4-phosphate and phosphoenolpyruvate, in a 9-step biosynthetic pathway which differs only with regard to the last-steps after prephenate synthesis. Tryptophan is also produced by these two starting molecules, but is synthesized in an 11-step pathway. Tyrosine can also be produced from phenylalanine in a reaction catalyzed by phenylalanine hydroxylase. Alanine, valine and leucine in each case are synthesis products of pyruvate, the end product of glycolysis. Aspartate is formed from oxalacetate, an intermediate of the citrate cycle. Asparagine, methionine, threonine and lysine are produced in each case by converting aspartate. Isoleucine is formed from threonine. Histidine is formed in a complex 9-step pathway starting from 5-phosphoribosyl-1-pyrophosphate, an activated sugar.

Amino acids whose quantity exceeds the cell's requirement for protein biosynthesis cannot be stored and are instead degraded so that intermediates are provided for the main metabolic pathways of the cell (for a review see Stryer, L., Biochemistry, 3rd Ed. Chapter 21 “Amino Acid Degradation and the Urea Cycle”; pp 495-516 (1988)). While the cell is capable of converting undesired amino acids into useful metabolic intermediates, amino acid production requires large amounts of energy, of precursor molecules and of the enzymes required for their synthesis. It is therefore not surprising that amino acid biosynthesis is regulated by feedback inhibition, the presence of a certain amino acid slowing down, or completely ending, its own production (for a review of the feedback mechanism in amino acid biosynthetic pathways, see Stryer, L., Biochemistry, 3rd Ed. Chapter 24, “Biosynthesis of Amino Acids and Heme”, pp. 575-600 (1988)). The output of a particular amino acid is therefore limited by the amount of this amino acid present in the cell.

B. Metabolism and Uses of Vitamins, Cofactors and Nutraceuticals

Vitamins, cofactors and neutraceuticals constitute a further group of molecules. How animals have lost the ability of synthesizing them and they therefore have to be ingested even though they are synthesized readily by other organisms such as bacteria. These molecules are either bioactive molecules per se or precursors of bioactive substances which act as electron carriers or intermediates in a series of metabolic pathways. Besides their nutritional value, these compounds also have a significant industrial value as colorants, antioxidants and catalysts or other processing auxiliaries. (For a review over the structure, activity and industrial applications of these compounds, see, for example, Ullmann's Encyclopedia of Industrial Chemistry, “Vitamins”, Vol. A27, pp. 443-613, VCH Weinheim, 1996). The term “vitamin” is known in the art and comprises nutrients which are required by an organism for its normal function, but which cannot be synthesized by this organism itself. The group of vitamins may comprise cofactors and nutraceutical compounds. The term “cofactor” comprises nonproteinaceous compounds which are required for a normal enzyme activity to occur. These compounds can be organic or inorganic; the cofactor molecules according to the invention are preferably organic. The term “nutroceutical” comprises food additives which are health-promoting in plants and animals, in particular humans. Examples of such molecules are vitamins, antioxidants and also certain lipids (for example polyunsaturated fatty acids).

The biosynthesis of these molecules in organisms which are capable of producing them, such as bacteria, has been characterized comprehensively (Ullmann's Encyclopedia of Industrial Chemistry, “Vitamins”, Vol. A27, pp. 443-613, VCH Weinheim, 1996, Michal, G. (1999) Biochemical Pathways: An Atlas of Biochemistry and Molecular Biology, John Wiley & Sons; Ong, A. S., Niki, E. and Packer, L. (1995) “Nutrition, Lipids, Health and Disease” Proceedings of the UNESCO/Confederation of Scientific and Technological Associations in Malaysia and the Society for free Radical Research—Asia, held on Sep. 1-3, 1994, in Penang, Malaysia, AOCS Press, Champaign, IL X, 374 pp).

Thiamine (vitamin B₁) is formed by chemically coupling pyrimidine and thiazole units. Riboflavin (vitamin B₂) is synthesized from guanosine-5′-triphosphate (GTP) and ribose-5′-phosphate. Riboflavin, in turn, is employed for the synthesis of flavin mononucleotide (FMN) and flavin adenine dinucleotide (FAD). The family of compounds which together are termed “vitamin B6” (for example pyridoxine, pyridoxamine, pyridoxal-5′-phosphate and pyridoxine hydrochloride, the latter being used commercially) are all derivatives of the structural unit 5-hydroxy-6-methylpyridine which they share. Panthothenate (pantothenic acid, R-(+)-N-(2,4-dihydroxy-3,3-dimethyl-1-oxobutyl)-β-alanine) can either be synthesized chemically or produced by fermentation. The last steps in pantothenate biosynthesis consist of the ATP-driven condensation of β-alanine and pantoic acid. The enzymes responsible for the biosynthesis steps for the conversion into pantoic acid and into β-alanine and for the condensation to give pantothenic acid are known. The metabolically active form of pantothenate is coenzyme A, whose biosynthesis involves 5 enzymatic steps. Pantothenate, pyridoxal-5′-phosphate, cysteine and ATP are the precursors of coenzyme A. These enzymes not only catalyze the formation of pantothenate, but also the production of (R)-pantoic acid, (R)-pantolactone, (R)-panthenol (provitamin B₅), pantethein (and its derivatives) and coenzyme A.

The biosynthesis of biotin from the precursor molecule pimeloyl-CoA in microorganisms has been studied extensively, and several of the genes involved have been identified. It has emerged that many of the proteins in question are involved in an Fe cluster synthesis and belong to the class of the nifS proteins. Lipoic acid is derived from octanoic acid and acts as a coenzyme in energy metabolism, where it enters the pyruvate dehydrogenase complex and the α-ketoglutarate dehydrogenase complex. The folates are a group of substances all of which are derived from folic acid, which, in turn, is derived from L-glutamic acid, p-aminobenzoic acid and 6-methylpterin. The biosynthesis of folic acid and its derivatives, starting from the metabolic intermediates guanosine-5′-triphosphate (GTP), L-glutamic acid and p-aminobenzoic acid, has been studied in detail in certain microorganisms.

Corrinoids (such as the cobalamines and, in particular, vitamin B₁₂) and the prophyrins belong to a group of chemicals distinguished by a tetrapyrrole ring system. The biosynthesis of vitamin B₁₂ is sufficiently complex so that it has not been characterized fully, but most of the enzymes and substrates involved are known by now. Nicotinic acid (nicotinate) and nicotinamide are pyridine derivatives also termed “niacin”. Niacin is the precursor of the important coenzymes NAD (nicotinamide adenine dinucleotide) and NADP (nicotinamide adenine dinucleotide phosphate) and of their reduced forms.

The production of these compounds on a large scale is mostly based on cell-free chemical syntheses, even though some of these chemicals have also been produced by culturing microorganisms on a large scale, such as riboflavin, vitamin B₆, pantothenate and biotin. Only vitamin B₁₂ is exclusively produced by fermentation, owing to the complexity of its synthesis. In-vitro methods require a great outlay of materials, are time-consuming and are frequently costly.

C. Metabolism and Uses of Purines, Pyrimidines, Nucleosides and Nucleotides

Genes for purine and pyrimidine metabolism and their corresponding proteins are important targets for the therapy of tumor diseases and viral infections. The term “purine” or “pyrimidine” comprises nitrogenous bases which constitute a component of the nucleic acids, enzymes and nucleotides. The term “nucleotide” encompasses the basic structural units of the nucleic acid molecules, which units comprise a nitrogenous base, a pentose sugar (the sugar being ribose in the case of DNA and D-deoxyribose in the case of DNA) and phosphoric acid. The term “nucleoside” comprises molecules which act as precursors of nucleotides but which, in contrast to the nucleotides, lack a phosphoric acid unit. Inhibiting the biosynthesis of these molecules or their mobilization for forming nucleic acid molecules makes it possible to inhibit RNA and DNA synthesis; if this activity is inhibited in a directed fashion in carcinogenic cells, the ability of tumor cells to divide and to replicate can be inhibited.

In addition, nucleotides exist which do not form nucleic acid molecules but which store energy (i.e. AMP) or which act as coenzymes (i.e. FAD and NAD).

Several publications have dealt with the use of these chemicals for these medical indications, where the purine and/or pyrimidine metabolism is affected (for example Christopherson, R. I. and Lyons, S. D. (1990) “Potent inhibitors of de novo pyrimidine and purine biosynthesis as chemotherapeutic agents”, Med. Res. Reviews 10: 505-548). Studies into enzymes which participate in purine and pyrimidine metabolism have centered on the development of novel drugs which can be used, for example, as immunosuppressants or antiproliferants (Smith, J. L. “Enzymes in Nucleotide Synthesis” Curr. Opin. Struct. Biol. 5 (1995) 752-757; Biochem. Soc. Transact. 23 (1995) 877-902). However, the purine and pyrimidine bases, nucleosides and nucleotides can also be used for other purposes: as intermediates in the biosynthesis of various fine chemicals (for example thiamine, S-adenosylmethionine, folate or riboflavin), as energy carriers for the cell (for example ATP or GTP) and for chemicals themselves, are usually used as flavor enhancers (for example IMP or GMP) or for a multiplicity of uses in medicine (see, for example, Kuninaka, A., (1996) “Nucleotides and Related Compounds in Biotechnology Vol. 6, Rehm et al., Ed. VCH Weinheim, pp. 561-612). Enzymes which are involved in the metabolism of purines, pyrimidines, nucleosides or nucleotides also increasingly act as targets against which crop protection chemicals including fungicides, herbicides and insecticides are being developed.

The metabolism of these compounds in bacteria has been characterized (for reviews see, for example, Zalkin, H. and Dixon, J. E. (1992) “De novo purine nucleotide biosynthesis” in Progress in Nucleic Acids Research and Molecular biology, Vol. 42, Academic Press, pp. 259-287; and Michal, G. (1999) “Nucleotides and Nucleosides”; Chapter 8 in: Biochemical Pathways: An Atlas of Biochemistry and Molecular Biology, Wiley, New York). Purine metabolism, the object of intense research, is essential for normal cell functioning. Defects in the purine metabolism in higher animals may cause severe diseases, for example gout. The purine nucleotides are synthesized starting from ribose-5-phosphate in a series of steps via the intermediate inosine-5′-phosphate (IMP), leading to the production of guanosine-5′-monophosphate (GMP) or adenosine-5′-monophosphate (AMP), and the triphosphate forms used as nucleotides can be prepared readily from these. These compounds are also used as energy stores such that their degradation yields energy for a variety of different biochemical processes in the cell. Pyrimidine biosynthesis takes place via the formation of uridine 5′-monophosphate (UMP) from ribose-5-phosphate. UMP, in turn is converted into cytidine-5′-triphosphate (CTP). The deoxy forms of all nucleotides are produced in a one-step reduction reaction from the diphosphate-ribose form of the nucleotide to give the diphosphate deoxyribose form of the nucleotide. After phosphorylation, these molecules can particulate in the synthesis of DNA.

D. Metabolism and Uses of Trehalose

Trehalose is composed of two glucose molecules which are linked to each other via an α,α-1,1 bond. It is normally used in the food industry as sweetener, as additive for dried or frozen foods, and in beverages. However, it is also used in the pharmaceuticals, cosmetics and biotechnology industries (see, for example, Nishimoto et al., (1998) U.S. Pat. No. 5,759,610; Singer, M. A. and Lindquist, S. Trends Biotech. 16 (1998) 460-467; Paiva, C. L. A. and Panek, A. D. Biotech Ann. Rev. 2 (1996) 293-314; and Shiosaka, M. J. Japan 172 (1997) 97-102). Trehalose is produced by many microorganisms using enzymes and released naturally into the surrounding medium, from which it can be recovered by processes known in the art.

This procedure may also be carried out analogously using other bacteria.

### EXAMPLE

Any sequence segment of the C. glutamicum ddh gene (Ishino et al.(1987) Nucleic Acids Res. 15, 3917), in particular a fragment in the 5′-terminal region of the coding region, can be amplified by PCR using known methods, and the resulting PCT product can be cloned into pSL18 ((Kim, Y. H. & H. -S. Lee (1996) J. Microbiol. Biotechnol. 6, 315-320), thus giving rise to vector pSL18Δddh. Other vectors which contain a marker gene which is suitable for C. glutamicum may also be used for this purpose. The skilled worker will be familiar with the procedure.

The cglIM gene can be expressed in different ways in a suitable E. coli strain (McrBC-deficient (alternative term: hsdRM-deficient), such as, for example NM522 or HB101), either as genomic copy of else on plasmids. One method consists in the use of plasmid pTc15AcglIM. Plasmid pTc15AcglIM comprises the origin of replication of plasmid p15A (Selzer et al. (1983) Cell 32, 119-129), a tetracycline resistance gene (Genbank Acc. No. J01749) and the cglIM gene (Schäfer et al. (1997) Gene 203, 93-101). E. coli strains which harbor pTc15AcglIM have DNA which carries the cglIM methylation pattern. Accordingly, the pSL18 derivatives (such as pSL18Δddh, see above) are also “cglIM methylated”.

The plasmid DNA of strain NM522(pTc15AcglIM/pSL18Δddh) can be prepared by customary methods (Sambrook, J. et al. (1989) “Molecular Cloning: A Laboratory Manual”. Cold Spring Harbor Laboratory Press or Ausubel, F. M. et al. (1994) “Current Protocols in Molecular Biology”, John Wiley & Sons) and this DNA can be employed for the electroporation of C. glutamicum (Liebl et al. (1989) FEMS Microbiol. Lett. 65, 299-304). C. glutamicum ATCC13032 may be used for this purpose, however, other corynebacteria may also be used.

In none of our experiments did plasmid pSL18Δddh, obtained from an E. coli strain without pTc15AcglIM, lead to transformants following electroporation. In contrast, pSL18Δddh, obtained from a pTc15AcglIM-harboring E. coli strain, allowed the recovery of transformants by electroporation. These transformants were clones in which the ddh gene was deactivated, as was shown, for example, by the absence of Ddh activity. Ddh activity can be measured by known methods (see, for example, Misono et al. (1986) Agric. Biol. Chem. 50, 1329-1330).

We claim:

1. A process for producing corynebacteria comprising one or more modified genomic sequences, where a vector is used which does not replicate in corynebacteria and whose nucleic acid is not recognized by corynebacteria as foreign.

2. A process as claimed in claim 1, the vector carrying the corynebacterial DNA methylation pattern.

3. A process as claimed in claim 2, the methylation pattern being obtainable by a methyl transferase.

4. A process as claimed in any of claims 1 to 3, the corynebacteria being Corynebacterium glutamicum, Corynebacterium ammoniagenes, Corynebacterium diphtheriae, Corynebacterium lactofermentum, Brevibacterium lactofermentum or Brevibacterium brevis.

5. A process as claimed in any of claims 1 to 4, the modified genomic sequences being one or more point mutations, one or more disruptions, and the introduction of one or more genes which are present in the organism or else foreign.

6. A process for the production of fine chemicals, a microorganism produced by one of the processes claimed in claims 1 to 5 being used for producing the fine chemical.

7. A process as claimed in claim 6, the fine chemical being a naturally occurring amino acid, in particular lysine, threonine, glutamate or methionine, or a vitamin, in particular riboflavin or pantothenic acid.

8. A process as claimed in any of claims 2 to 7, the methylation pattern being obtainable by methyl transferase cglIM.

9. A vector which does not replicate in corynebacteria and which has a corynebacteria-specific methylation pattern.

10. A vector as claimed in claim 9 with a methylation pattern obtainable by a methyl transferase, in particular cglIM.
